(19) United States
(12) Patent Application Publication
(10) Pub. No.: US 2012/0000603 A1
(43) Pub. Date: Jan. 5, 2012

(54) ADHESIVE

(75) Inventors: Christos Karafilidis, Leverkusen (DE); Matthias Wintermantel, Bergisch Gladbach (DE); Heinz-Werner Lucas, Bergisch Gladbach (DE)

(73) Assignee: Bayer Materialscience AG, Leverkusen (DE)

(21) Appl. No.: 13/201,325

(22) PCT Filed: Feb. 2, 2010
(86) PCT No.: PCT/EP10/00617

§ 371 Date: Sep. 20, 2011

(30) Foreign Application Priority Data
Feb. 13, 2009 (DE) 10 2009 008 867.9

(51) Int. Cl. C09J 4/04; C09J 175/06; A61L 24/04; C07C 271/22

(52) U.S. Cl. 156/331.7; 560/158; 528/65; 528/61

(57) ABSTRACT

The invention relates to the use of special isocyanate-terminated polyurethane prepolymers in adhesive formulations. Said adhesive formulations can be used in applications wherein a direct or indirect contact of the adhesive layer takes place with substrates that are sensitive thereto.

The invention relates to the use of special isocyanate-terminated polyurethane prepolymers in adhesive formulations. These adhesive formulations can be used in applications in which it is important to avoid or minimise migrates in direct or indirect contact of the adhesive layer with substrates that are sensitive thereto.

These sensitive substrates can be, for example, human skin or composite films. The latter are widely used to produce packaging for all kinds of goods. Since it is not possible for all requirements, such as transparency/opacity, printability, barrier properties, sealability and mechanical properties, to be covered by monofilms, co-extruded multi-layer films or extrusion-laminated film composites, composite films in which the individual layers are bonded together using adhesive make up the largest share of the market and thus have immense commercial importance.

The production of food packaging from composite films is particularly significant. Since, on the side facing the food, some of the layers used have low barrier properties against the adhesive components employed, particular attention must be paid to any migration of adhesive components into the food.

In surgery, adhesives are increasingly being used for wound closure and care. It is particularly important in this case that no harmful substances migrate from the adhesive layer into the skin or the system.

In the area of flexible composite packaging films, aromatic polyurethane systems are predominantly used. The migration of aromatic polyisocyanates, or their reaction products with water, into the food is therefore particularly critical. With water, which is contained in almost all foods, polyisocyanates react with the release of carbon dioxide to form primary aromatic amines (PAAs). Since PAAs are toxic, the legislator has issued limits for migrates from food packaging, which it is imperative to observe. For this reason, the adhesives used for the production of composite films must have cured sufficiently fully when the food is packed so that migration is safely below the limits.

After their production, therefore, the composite films must be stored before packing the food until the reaction has progressed so far that no more migration of PAAs can be detected or the migration falls below the prescribed limits. To test for the migration of PAAs, the method according to section 64 LFGB (German Food and Feed Code) is generally used. To this end, a pouch made of the film composite to be tested is filled with a food simulant (usually 3 wt. % aqueous acetic acid solution), stored for 2 h at 70° C. and then the PAA content is tested photometrically after derivatisation. Contents of less than 0.2 μg PAAs per 100 ml of food simulant must be achieved. This corresponds to 2 ppb and, at the same time, the limit of detection of the method described. In the following text, the expression “freedom from migrates” or “migrate-free film composites” is used when migration is below this limit.

For both economic and logistic reasons, attempts are naturally being made to minimise the storage time necessary to achieve freedom from migrates. Two different concepts are being employed to this end:
1) Raw materials are used which contain only small quantities of aromatic isocyanates that are capable of migrating, i.e. monomers.
2) The chemical curing reaction of the adhesive formulation is accelerated.

EP-A 0 590 398 describes the use of low-monomer, isocyanate-terminated polyurethane prepolymers, which have been obtained by removal of the monomeric polyisocyanates by distillation, in solvent-free, 2-pack adhesive formulations for the production of flexible film composites. The film composites thus produced are free from migrates within three days, determined by the method according to section 64 LFBG. This procedure requires, in addition to the synthesis of the isocyanate-terminated crude polyurethane prepolymer, a time-consuming distillation step which increases production costs and cannot be carried out using conventional stirred vessels without system design changes. Moreover, the viscosity of the low-monomer, isocyanate-terminated polyurethane prepolymers is higher than that of conventional isocyanate-terminated polyurethane prepolymers. For example, low-monomer diphenylmethane diisocyanate polyurethane prepolymers with an isocyanate content of >6 wt. % have a viscosity of >10,000 mPas at 50° C. This viscosity is too high for application in adhesive formulations for flexible packaging, however. Moreover, the content of monomeric polyisocyanate has to be monitored, which means increased logistic and financial costs.

From DE-A 4 136 490, the use of asymmetric polyisocyanates with NCO groups of different reactivity (e.g. 2,4-toluene diisocyanate) is known. As a result of the different reactivity of the isocyanate groups, it is possible to produce low-monomer, isocyanate-terminated polyurethane prepolymers in a one-step process without removing the monomer by distillation. These are then used in solvent-free 2-pack adhesive formulations for the production of flexible film composites, which are migrate-free within three days. However, the viscosity of the low-monomer isocyanate-terminated polyurethane prepolymers is very high and the content of monomeric polyisocyanate has to be monitored, which means increased logistic and financial costs

DE-A 3 401 129 describes the production of low-monomer isocyanate-terminated polyurethane prepolymers in a 2-step process using at least two polyisocyanates having different reactivity (e.g. toluene diisocyanate and diphenylmethane diisocyanate). In addition to the use of the low-monomer prepolymers, the use of a “conventional accelerator” is disclosed. As an application, the use of the low-monomer prepolymers in adhesive formulations for bonding films is described. Disadvantages here are the use and metering of two isocyanates with different reactivity and the need to monitor the content of monomeric polyisocyanate.

U.S. 2006/0078741 describes the use of catalysts to reduce the curing time of adhesive formulations for the production of film composites. The shorter curing time correlates to the storage time that is needed in order to obtain a migrate-free film composite. Disadvantages of the use of a catalyst are its ability to migrate and the undesired heavy metal content in the catalysts, which are generally metallic.

G. Henke in Coating, March 2002 p. 90 ff. describes the prior art and explains that the latest generation of adhesive formulations for the production of film composites are migrate-free after a three-day storage period following lamination.

In DE-A 102 008 009 407 we described the use of isocyanate-terminated polyurethane prepolymers which contain tertiary amino groups in adhesive formulations for the production of film composites which give migrate-free film composites after no more than three days, and in the production of medical wound care systems.

It has now been found that, by using an isocyanate-terminated polyurethane prepolymer, which is not necessarily low in monomers but which contains tertiary amino groups and ethylene oxide in the polyol used to produce the polyurethane prepolymer, in an adhesive formulation with a polyol or a polyol mixture, adhesive preparations are obtained which can be used advantageously. These are suitable for the production of, among other things, adhesive bonds from which it is important that no monomers diffuse out, because they come into contact with the skin or with foods, for example. In a preferred use, the adhesive preparations according to the invention are used e.g. for the production of composite films, which are migrate-free after three days or sooner in accordance with section 64 LFGB. In another preferred use, adhesive preparations according to the invention are used as surgical adhesives for wound closure and care or in the production of adhesive and plaster systems for wound closure and care, as known e.g. from EP-A 0 897 406 as plasters, or without a textile support directly as a wound adhesive or wound closure means. In addition, active ingredients having a positive effect on wound behaviour may be incorporated into these adhesive preparations. These include, for example, agents having an antimicrobial action, such as antimycotics and substances having an antibacterial action (antibiotics), corticosteroids, chitosan, dexpanthenol and chlorhexidine gluconate.

The present invention therefore relates to the use of isocyanate-terminated polyurethane prepolymers containing tertiary amino groups and ethylene oxide in the polyol used to produce the polyurethane prepolymer in adhesive formulations for the production of film composites which give migrate-free film composites after no more than three days, and in the production of medical wound care systems.

It is advantageous in relation to the prior art and the publication DE-A 102 008 009 407 that, in contrast to the prior art, the production of the isocyanate-terminated prepolymers is possible in a 1-step process in a conventional stirred vessel, without expensive distillation, without the use of an asymmetrical isocyanate (which is not always available) and without quality control of the content of monomeric polyisocyanate, and leads to migrate-free film composites after the same or a shorter period. Furthermore, the isocyanate-terminated polyurethane prepolymers according to the invention exhibit lower viscosity compared with the low-monomer isocyanate-terminated polyurethane prepolymers of the prior art described above, and it is not necessary to add a catalyst, which is usually capable of migration, reduces storage life and is undesirable in food packaging because of its possible heavy metal content.

The present invention accordingly provides preferably the use of an isocyanate-terminated polyurethane prepolymer containing tertiary amino groups and ethylene oxide in adhesive formulations, which are migrate-free after three days and are used particularly preferably for the production of film composites. The polyurethane prepolymer and the adhesive formulation preferably display the following features:

The adhesive formulation preferably consists of an isocyanate-terminated polyurethane prepolymer A) and a polyol or polyol formulation B) and optionally other additives C).

A) The isocyanate-terminated polyurethane prepolymer
is a reaction product of a polyisocyanate or a polyisocyanate formulation a) and at least one polyol or polyol mixture b):
   a) The polyisocyanate or the polyisocyanate formulation
   generally contains polyisocyanates with a functionality of 2 to 3.5, preferably of 2 to 2.7, particularly preferably of 2 to 2.2 and most particularly preferably of 2, and an NCO content of 21 to 50 wt. %, preferably of 21 to 49 wt. %, particularly preferably of 29-34 wt. % and most particularly preferably of 33.6 wt. %.
   b) The polyol or polyol mixture
   generally contains at least one polyether, which contains tertiary amino groups, has a number-average molecular weight Mₙ of 320 to 20000 g/mol, preferably of 330 to 4500 g/mol, particularly preferably of 340 to 4200 g/mol and most particularly preferably of 3400 to 4100 g/mol and a nominal functionality of 2 to 4.5, preferably of 2.5 to 4.5, particularly preferably of 3 to 4.5 and most particularly preferably of 4, and optionally contains one or more additional polyethers and/or polyesters and/or polycarbonates with an average molecular weight Mₙ of 300 to 20000 g/mol, preferably of 430 to 17300 g/mol, particularly preferably of 590 to 8000 g/mol and most particularly preferably of 1000 to 4000 g/mol.
   The polyol or polyol mixture preferably has the following features:
      1. The polyol contains structural elements of the formula —CH₂—CH₂—O— and to produce this polyol, ethylene oxide was used exclusively or in a proportion as one of the monomers employed, or one or more polyols in the polyol mixture contain structural elements of the formula —CH₂—CH₂—O— and to produce these polyols, ethylene oxide was used exclusively or in a proportion as one of the monomers employed.
      2. The proportion of ethylene oxide used in the production of the polyols containing structural elements of the formula —CH₂—CH₂—O— is, based on the quantity of monomers used, i.e. excluding the initiator, between 10 and 100 wt. %, preferably between 20 and 100 wt. % and particularly preferably between 30 and 100 wt. %. Most particularly preferably, the ethylene oxide content, based on the quantity of monomers used, i.e. excluding the initiator, in the polyol not containing tertiary amino groups is 40 to 100 wt. % and the ethylene oxide content of the polyol containing tertiary amino groups is 0-20 wt. %.

B) This polyol or polyol formulation:
a) has a hydroxyl value of 40 to 300 mg KOH/g, preferably of 80 to 270 mg KOH/g and particularly preferably of 180 to 240 mg KOH/g,
b) has a nominal average functionality of 2 to 4, preferably 2 to 3.4 and particularly preferably of 2 to 2.9,
c) is a polyol, polyether polyol, polycarbonate polyol, polyether ester polyol or a polyester polyol or a mixture of two or more of said polyols,
d) can be produced from a proportion of ethylene oxide as one of the monomers used, with a content of ethylene oxide, based on the quantity of the monomers used, i.e. excluding the initiator, between 10 and 100 wt. %, preferably between 20 and 100 wt. %, and particularly preferably between 30 and 100 wt. %.

C) Optionally other additives, such as for example fillers, catalysts or viscosity adjusters.

To produce the ready-to-use adhesive formulation, the components A) and B) are mixed in a molar ratio of isocyanate groups:hydroxyl groups of 1:1 to 1.8:1, preferably in a molar ratio of isocyanate groups:hydroxyl groups of 1:1 to 1.6:1 and particularly preferably in a molar ratio of isocyanate groups:hydroxyl groups of 1.05:1 to 1.5:1.

The isocyanate-terminated polyurethane prepolymer A) is characterised in that it
a) has an NCO content of 5-20 wt. %, preferably an NCO content of 9-19 wt. %, particularly preferably an NCO content of 12-18 wt. % and most particularly preferably an NCO content of 13-17 wt. %,
b) has a nominal average functionality of 2 to 3, preferably of 2 to 2.7, particularly preferably of 2 to 2.4 and most particularly preferably of 2 to 2.1.

The production of isocyanate-terminated and tertiary amino group-containing polyurethane prepolymers A) is known per se to the person skilled in the art from polyurethane chemistry. The reaction of the components A) a) and A) b) in the production of the polyurethane prepolymers A) takes place e.g. by mixing the polyols, which are liquid at reaction temperatures, with an excess of the polyisocyanates and stirring the homogeneous mixture until a constant NCO value is obtained. A reaction temperature of 40° C. to 180° C., preferably 50° C. to 140° C., is selected. The production of the polyurethane prepolymers A) can also, of course, take place continuously in a stirred vessel cascade or in suitable mixing equipment, such as e.g. high-speed mixers according to the rotor-stator principle.

The following polyisocyanates, for example, are suitable for the production of isocyanate-terminated polyurethane prepolymers A):

1,6-hexamethylene diisocyanate (HDI), 1-isocyanatomethyl-3-isocyanato-1,5,5-trimethylcyclohexane (isophorone diisocyanate, IPDI), xylylene diisocyanate (XDI), dicyclohexylmethane-4,4′-diisocyanate (H12-MDI), 2,4- and 2,6-toluene diisocyanate (TDI), diphenylmethane 2,2′-diisocyanate, diphenylmethane 2,4′-diisocyanate, diphenyl-methane 4,4′-diisocyanate (MDI) or mixtures of two or more of said polyisocyanates, as well as oligomers thereof.

Preferably, diphenylmethane 2,2′-diisocyanate, diphenylmethane 2,4′-diisocyanate and diphenylmethane 4,4′-diisocyanate (MDI) and mixtures thereof are used to produce component A).

Particularly preferably, a mixture of max. 1 wt. % diphenylmethane 2,2′-diisocyanate, 40 to 70 wt. % diphenylmethane 2,4′-diisocyanate and 28 to 60 wt. % diphenylmethane 4,4′-diisocyanate (MDI) is used to produce component A).

Most particularly preferably, a mixture of max. 0.2 wt. % diphenylmethane 2,2′-diisocyanate, 50 to 60 wt. % diphenylmethane 2,4′-diisocyanate and at least 38.5 wt. % diphenylmethane 4,4′-diisocyanate (MDI) is used to produce component A).

To produce isocyanate-terminated polyurethane prepolymers A) and adhesive formulations B), for example the following polyols can be used:

Polyether polyols suitable for the production of the isocyanate-terminated polyurethane prepolymer A) and the polyol formulation B) are known per se to the person skilled in the art from polyurethane chemistry. These are typically obtained starting from low-molecular-weight, polyfunctional, OH- or NH-functional compounds as initiators by reaction with cyclic ethers or mixtures of different cyclic ethers. As catalysts here, bases such as KOH or double metal cyanide-based systems are used. Production processes that are suitable for this purpose are known per se to the person skilled in the art e.g. from U.S. Pat. No. 6,486,361 or L. E. St. Pierre, Polyethers Part I, Polyalkylene Oxide and other Polyethers, Editor: Norman G. Gaylord; High Polymers Vol. XIII; Interscience Publishers; Newark 1963; p. 130 ff.

These are, for example:

Polyether polyols which contain tertiary amino groups and are suitable for use as polyol component ii) for the production of the isocyanate-terminated polyurethane prepolymer A) can be produced from a large number of aliphatic and aromatic amines which contain one or more primary or secondary amino groups. As initiators for the production of the tertiary amino group-containing polyethers, for example the following amino compounds or mixtures of these amino compounds can be used: ammonia, methylamine, triethanolamine, N-methyldiethanolamine, N,N,-dimethylethanolamine, ethylenediamine, N,N-dimethylethylenediamine, N,N′-dimethylethylenediamine, tetramethylenediamine, hexamethylene-diamine, 2,4-toluenediamine, 2,6-toluenediamine, aniline, diphenylmethane-2,2′-diamine, diphenylmethane-2,4′-diamine, diphenylmethane-4,4′-diamine, 1-aminomethyl-3-amino-1,5,5-trimethylcyclohexane (isophorone diamine), dicyclohexylmethane-4,4′-diamine and xylylenediamine.

Particularly preferred are the amines ethylenediamine, N,N-dimethylethylenediamine, N,N′-dimethylethylenediamine, triethanolamine and N-methyldiethanolamine.

In a particularly preferred exemplary embodiment, ethylenediamine is used.

Polyether polyols that do not contain any tertiary amino groups and are suitable for use as polyol component ii) for the production of the isocyanate-terminated polyurethane prepolymer A) or for use in the polyol formulation B) can be produced from a large number of alcohols which contain one or more primary or secondary alcohol groups. As initiators for the production of the polyethers containing no tertiary amino groups, the following compounds, for example, or mixtures of these compounds, may be used: water, ethylene glycol, propylene glycol, glycerol, butanediol, butanetriol, trimethylolethane, pentaerythritol, hexanediol, 3-hydroxyphenol, hexanetriol, trimethylolpropane, octanediol, neopentyl glycol, 1,4-hydroxymethylcyclohexane, bis(4-hydroxyphenyl) dimethylmethane and sorbitol. Ethylene glycol, propylene glycol, glycerol and trimethylolpropane are preferably used, particularly preferably ethylene glycol and propylene glycol, and in a particularly preferred exemplary embodiment propylene glycol is used.

Suitable as cyclic ethers for the production of the polyethers described above are alkylene oxides, such as ethylene oxide, propylene oxide, butylene oxide, epichlorohydrin, styrene oxide or tetrahydrofuran, or mixtures of these alkylene oxides. The use of propylene oxide, ethylene oxide or tetrahydrofuran or mixtures of these is preferred. Propylene oxide or ethylene oxide or mixtures of these are particularly preferably used. Propylene oxide is most particularly preferably used.

The polyester polyols suitable for the production of the isocyanate-terminated polyurethane prepolymer A) and the polyol formulation B) are known per se to the person skilled in the art from polyurethane chemistry.

Thus, for example, it is possible to produce polyester polyols which are formed by the reaction of low-molecular-weight alcohols, particularly of ethylene glycol, diethylene glycol, neopentyl glycol, hexanediol, butanediol, propylene glycol, glycerol or trimethylolpropane with caprolactone. Also suitable as polyfunctional alcohols for the production of polyester polyols are 1,4-hydroxymethylcyclohexane, 2-methyl-1,3-propanediol, 1,2,4-butanetriol, triethylene glycol, tetraethylene glycol, polyethylene glycol, dipropylene glycol, polypropylene glycol, dibutylene glycol and polybutylene glycol.

Other suitable polyester polyols can be produced by polycondensation. For example, difunctional and/or trifunctional alcohols can be condensed with a substoichiometric amount of dicarboxylic acids or tricarboxylic acids or mixtures of dicarboxylic acids or tricarboxylic acids, or the reactive derivatives thereof, to form polyester polyols. Suitable dicarboxylic acids are, for example, adipic acid or succinic acid and their higher homologues with up to 16 C atoms, and also unsaturated dicarboxylic acids, such as maleic acid or fumaric acid, as well as aromatic dicarboxylic acids, particularly the isomeric phthalic acids, such as phthalic acid, isophthalic acid or terephthalic acid. Suitable tricarboxylic acids are e.g. citric acid or trimellitic acid. The above acids may be used individually or as mixtures of two or more thereof. Particularly suitable alcohols are hexanediol, butanediol, ethylene glycol, diethylene glycol, neopentyl glycol, 3-hydroxy-2,2-dimethylpropyl-3-hydroxy-2,2-dimethylpropanoate or trimethylolpropane or mixtures of two or more thereof. Particularly suitable acids are phthalic acid, isophthalic acid, terephthalic acid, adipic acid or dodecanedioic acid or mixtures thereof.

Polyester polyols with a high molecular weight include, for example, the reaction products of polyfunctional, preferably difunctional alcohols (optionally together with small amounts of trifunctional alcohols) and polyfunctional, preferably difunctional carboxylic acids. Instead of free polycarboxylic acids, (if possible) the corresponding polycarboxylic anhydrides or corresponding polycarboxylic acid esters with alcohols having preferably 1 to 3 C atoms may be used. The polycarboxylic acids may be aliphatic, cycloaliphatic, aromatic or heterocyclic or both. They may optionally be substituted, e.g. by alkyl groups, alkenyl groups, ether groups or halogens. Suitable polycarboxylic acids are e.g. succinic acid, adipic acid, suberic acid, azelaic acid, sebacic acid, dodecanedioic acid, phthalic acid, isophthalic acid, terephthalic acid, trimellitic acid, phthalic anhydride, tetrahydrophthalic anhydride, hexahydrophthalic anhydride, tetrachlorophthalic anhydride, endomethylene tetrahydrophthalic anhydride, glutaric anhydride, maleic acid, maleic anhydride, fumaric acid, dimer fatty acid or trimer fatty acid or mixtures of two or more thereof.

It is also possible to use polyesters obtainable from lactones, e.g. based on ε-caprolactone, also known as “polycaprolactone”, or hydroxycarboxylic acids, e.g. ω-hydroxycaproic acid.

However, it is also possible to use polyester polyols of oleochemical origin. These polyester polyols can be produced e.g. by complete ring opening of epoxidised triglycerides of an at least partially olefinically unsaturated fatty acid-containing fat mixture with one or more alcohols having 1 to 12 C atoms and subsequent partial transesterification of the triglyceride derivatives to form alkyl ester polyols having 1 to 12 C atoms in the alkyl radical.

The polycarbonate polyols suitable for the production of the isocyanate-terminated polyurethane prepolymer A) and the polyol formulation B) are known per se to the person skilled in the art from polyurethane chemistry.

Thus, for example, it is possible to produce polycarbonate polyols by the reaction of diols, such as propylene glycol, 1,4-butanediol or 1,6-hexanediol, diethylene glycol, triethylene glycol or tetraethylene glycol or mixtures of these diols with diaryl carbonates, e.g. diphenyl carbonates, or phosgene.

Other additives C):

The adhesive formulation may also contain, in addition to the above-mentioned components, additives C) known from adhesives technology as formulation auxiliaries. These additives are e.g. the conventional plasticisers, fillers, pigments, drying agents, light stabilisers, antioxidants, thixotropic agents, adhesion promoters and optionally other auxiliary substances and additives.

Examples of suitable fillers that may be mentioned are carbon black, precipitated silicas, pyrogenic silicas, mineral chalks and precipitated chalks.

Suitable plasticisers are e.g. phthalic acid esters, adipic acid esters, alkylsulfonic acid esters of phenol or phosphoric acid esters.

Examples of thixotropic agents that may be mentioned are pyrogenic silicas, polyamides, hydrogenated castor oil derivatives or polyvinyl chloride.

Suitable drying agents are in particular alkoxysilyl compounds, such as e.g. vinyltrimethoxysilane, methyltrimethoxysilane, methyltriethoxysilane, i-butyltrimethoxy-silane, i-butyltriethoxysilane, octyltriethoxysilane, octyltrimethoxysilane, propyltriethoxy-silane, propyltrimethoxysilane, hexadecyltrimethoxysilane, and inorganic substances such as e.g. calcium oxide (CaO) and isocyanate group-containing compounds such as e.g. tosyl isocyanate.

The known functional silanes are used as adhesion promoters, such as e.g. aminosilanes of the aforementioned type, but also N-aminoethyl-3-aminopropyltrimethoxysilane, N-amino-ethyl-3-aminopropylmethyldimethoxysilane, N-aminoethyl-3-aminopropyltrimethoxysilane, 3-glycidoxypropyltrimethoxysilane, 3-glycidoxypropyltriethoxysilane, mercaptosilanes, bis(3-triethoxysilylpropyl)amine, bis(3-trimethoxysilylpropyl)amine, oligoaminosilanes, 3-aminopropylmethyldiethoxysilane, 3-aminopropyltriethoxysilane, 3-aminopropyltriethoxysilane, triamino-functional propyltrimethoxysilane, N-(n-butyl)-3-aminopropyltrimethoxysilane, phenyltriethoxysilane, phenyltrimethoxysilane, polyether-functional trimethoxysilanes and 3-methacryloxypropyltrimethoxysilane.

The method in principle for the production of the adhesive formulation from the isocyanate-terminated and tertiary amino group-containing polyurethane prepolymer A) and the polyol or polyol mixture B) and for the production of a film composite is known per se to the person skilled in the art from polyurethane chemistry.

The additives C) may be added to the polyol or polyol formulation B) or to the isocyanate-terminated and tertiary amino group-containing polyurethane prepolymer A) or both. Preferably, the additives C) are added to the polyol or polyol formulation B).

In one embodiment of the invention, the two components A) and B) of the adhesive formulation, to which the additives C) have optionally already been added, are mixed together immediately before the production of the film composite and introduced into the laminating machine or the applicator unit. In another embodiment of the invention, the mixing of the components A) and B), to which the additives C) have optionally already been added, may take place in the laminating machine itself immediately before or in the applicator unit.

The adhesive formulation may be used here as a 100% system, i.e. without solvents, or in a suitable solvent or a suitable solvent mixture for the production of the film composite.

In the applicator unit, the so-called support film is coated with the adhesive formulation with an average dry application weight of 1 to 9 g/m² and, by bringing it into contact with a second film, it is laminated to form the resulting film composite. If suitable solvents or solvent mixtures are used, the solvents must be removed completely in a drying tunnel or in another suitable device before the support film is brought into contact with the second film.

The adhesive formulation is preferably used for bonding plastics films, aluminium foils, other metal foils, plastics films with metal coatings and plastics films with metal oxide coatings.

The invention is explained by the following, non-restrictive examples.

### EXAMPLES

In the following examples, percentages refer to the weight.

Unless otherwise specified, the viscosities were determined at a measuring temperature of 25° C. with the aid of the Viscotester VT 550 rotational viscometer from Thermo Haake, Karlsruhe, Del. with the SV measuring cup and the SV DIN measuring device.

The NCO content of the prepolymers or reaction mixtures was determined in accordance with DIN EN 1242.

The monomer migration of aromatic polyisocyanates is determined on the basis of the method according to section 64 LFBG (method: BVL L 00.00-6 “Investigation of foodstuffs—Determination of primary aromatic amines in aqueous food simulants” from the collection of methods of the German Federal Office of Consumer Protection and Food Safety). The film composite to be investigated (polyethylene terephthalate/aluminium foil/polyethylene film) is stored as a roll sample under standard climatic conditions at 23° C. and 50% rel. humidity. After 1, 3 and 7 days, 5 layers of film web are unwound in each case and two test pieces each of approx. 120 mm×220 mm are removed to produce the test pouches. The test pouches (internal measurements 100 mm×200 mm) with the polyethylene film on the inside of the pouch are filled with 200 ml 3% aqueous acetic acid solution as food simulant, welded and stored for two hours at 70° C. Immediately after storage, the pouches are emptied and the food simulant solution is cooled to room temperature.

Detection of the migrated polyisocyanates takes place by diazotising the primary aromatic amines formed from the aromatic polyisocyanates in the aqueous food simulant and then coupling with N-(1-naphthy)ethylenediamine. For quantitative determination, the extinction values of the coupling component are measured against the respective zero sample, and the values are converted using a calibration curve to μg aniline hydrochloride/100 ml food simulant.

The following abbreviations are used:

OHV: Hydroxyl value [mg KOH/g]

AV: Acid value [mg KOH/g]

% NCO: NCO content in wt. % NCO groups

IA: Interlayer adhesion [N/15mm] between the aluminium and the polyethylene layer in the following composite 12 μm polyethylene terephthalate/9 μm aluminium foil/60 μm polyethylene film

SBS: Seal bond strength [N/15mm] of the seal of the polyethylene internal side of the film composite to itself (sealing temperature: 120° C., sealing time: 2 s, hot on both sides with smooth sealing bars)

MIG: Migrated polyisocyanates converted to pig aniline hydrochloride/100 ml food simulant [μg aniline hydrochloride/100 ml food simulant]

Abbreviations of reagents used:

### Polyols

P1: Polypropylene ether glycol, produced by KOH catalysis, OHV 112

P2: Polypropylene ether tetraol initiated with ethylenediamine, produced by KOH catalysis, OHV 60

P3: Polyester polyol as a reaction product of adipic acid and diethylene glycol, OHV 112, AV≦1.3

P4: Polyester polyol as a reaction product of adipic acid and diethylene glycol, OHV 43, AV≦1.5

P5: Polyester polyol as a reaction product of adipic acid as acid component and a mixture of 1 part by weight trimethylolpropane and 12.8 parts by weight diethylene glycol as alcohol component, OHV 60, AV≦2

P6: Trimethylolpropane, OHV 1250

P7: Diethylene glycol, OHV 1050

P8: Polypropylene ether glycol, produced by double metal cyanide catalysis, OHV 10

P9: Polyether glycol, produced by KOH catalysis, containing approx. 3.8 wt. % propylene glycol as initiator and ethylene oxide (EO) and propylene oxide (PO) in a weight ratio of 49:51 (EO:PO), OHV 57

P10: Polyethylene ether glycol, produced by KOH catalysis, OHV 56

### Polyisocyanates

NCO1: A mixture of 0.1% diphenylmethane 2,2′-diisocyanate, 50.8% diphenylmethane 2,4′-diisocyanate, 49.1% diphenylmethane 4,4′-diisocyanate

### Prepolymer Containing Tertiary Amino Groups Not According To the Invention

A polyol mixture of 1102 g P1 and 1102 g P2 is dehydrated by stirring for 1 hour at 120° C. under a vacuum of 20 mbar. It is then cooled to 70° C. The polyol mixture obtained is metered into 2797 g NCO1 within approx. 30 minutes. Then, utilising any exothermic reaction that may occur, it is heated to 80° C. and stirred for 2 h. It is stirred at 80° C. until the isocyanate content is constant. This results in an isocyanate-terminated polyurethane prepolymer with a content of 15.2% NCO and a viscosity of 1630 mPas (25° C.).

### Prepolymer 1 Containing Tertiary Amino Groups And Ethylene Oxide According To the Invention

A polyol mixture of 2550 g P2 and 2550 g P9 is dehydrated by stirring for 1 hour at 120° C. under a vacuum of 20 mbar. It is then cooled to 50° C. 5900 g NCO1 are metered into the polyol mixture obtained within approx. 30 minutes. Then, utilising any exothermic reaction that may occur, it is heated to 80° C. and stirred for 2 h. It is stirred at 80° C. until the isocyanate content is constant. This results in an isocyanate-terminated polyurethane prepolymer with a content of 15.8% NCO and a viscosity of 1160 mPas (25° C.).

### Prepolymer 2 Containing Tertiary Amino Groups And Ethylene Oxide According To the Invention

A polyol mixture of 346 g P2 and 346 g P10 is dehydrated by stirring for 1 hour at 120° C. under a vacuum of 20 mbar. It is then cooled to 50° C. The polyol mixture obtained is metered into 807 g NCO1 within approx. 30 minutes. Then, utilising any exothermic reaction that may occur, it is heated to 80° C. and stirred for 2 h. It is stirred at 80° C. until the isocyanate content is constant. This results in an isocyanate-terminated polyurethane prepolymer with a content of 16.2% NCO and a viscosity of 1150 mPas (23° C.).

### Prepolymer 3 Containing Tertiary Amino Groups And Ethylene Oxide According To the Invention

A polyol mixture of 426 g P2 and 426 g P10 is dehydrated by stirring for 1 hour at 120° C. under a vacuum of 20 mbar. It is then cooled to 50° C. The polyol mixture obtained is metered into 649 g NCO1 within approx. 30 minutes. Then, utilising any exothermic reaction that may occur, the mixture is heated to 80° C. and stirred for 2 h. It is stirred at 80° C. until the isocyanate content is constant. This results in an isocyanate-terminated polyurethane prepolymer with a content of 11.7% NCO and a viscosity of 3500 mPas (23° C.).

### Preparation of the Adhesive Formulation

Since the mixture of the polyol component and the polyisocyanate component is by nature unsuitable for storage, this is produced immediately before production of the film composite by intimate mixing of the polyol component and the polyisocyanate component and is processed immediately.

It is produced with a 1.4-times molar excess of isocyanate groups.

### Production of the Film Composites Using the Adhesive Formulations Described In Table 1

The film composites are produced using a “Polytest 440” solvent-free laminating unit from Polytype in Freiburg, Switzerland.

The film composites are produced from a polyethylene terephthalate/aluminium precomposite and a polyethylene film. The aluminium side of the precomposite is coated with the adhesive formulation, bonded with the polyethylene film and then wound on to a roll core. The length of the film composite produced with the adhesive formulation is at least 20 m. The dry application quantity of the adhesive formulation is between 1.9 g and 3.3 g and the roll temperature of the applicator unit is 30-50° C.

TABLE 1
Formulae and test results of the adhesive formulations:

| | | | | | Adhesive | | |
|---|---|---|---|---|---|---|---|
| | Adhesive formulation | | | | formulation | | |
| | not according | | | | according to the | | |
| | to the invention | | | | invention | | |
| Reagents in wt. % | 1* | 2* | 3* | 4* | 1 | 2 | 3 |
| Prepolymer not | 61.2 | 52.2 | 57.2 | 57.2 | | | |
| according to the | | | | | | | |
| invention containing | | | | | | | |
| exclusively | | | | | | | |
| tertiary amino groups | | | | | | | |
| Prepolymer 1 | | | | | 57.1 | | |
| according to the | | | | | | | |
| invention containing | | | | | | | |
| tertiary amino groups | | | | | | | |
| and ethylene oxide | | | | | | | |
| Prepolymer 2 | | | | | | 57.4 | |
| according to the | | | | | | | |
| invention containing | | | | | | | |
| tertiary amino groups | | | | | | | |
| and ethylene oxide | | | | | | | |
| Prepolymer 3 | | | | | | | 65.7 |
| according to the | | | | | | | |
| invention containing | | | | | | | |
| tertiary amino groups | | | | | | | |
| and ethylene oxide | | | | | | | |
| P3 | 34.7 | 26.4 | 3.5 | 6.1 | 39.7 | 39.5 | 31.8 |
| P4 | | 13.6 | 10.6 | 31.6 | | | |
| P5 | | | 23.8 | | | | |
| P6 | 3.1 | 3.3 | 4.9 | 5.1 | 3.2 | 3.2 | 2.5 |
| P7 | 1.0 | | | | | | |
| P8 | | 4.5 | | | | | |
| IA after x d | | | | | | | |
| 1 | 3.7 | 2.6 | 3.5 | 3.1 | 2.9 | 2.8 | 1.8 |
| 3 | 4.6 | 4.5 | 3.1 | 3.2 | 2.5 | 2.7 | 2.2 |
| 7 | 3.8 | 3.9 | 3.4 | 2.9 | 2.6 | 2.7 | 2.5 |
| SBS after x d | | | | | | | |
| 1 | 21.4 | 18.3 | 30.5 | 21.3 | 23.8 | 22.0 | 20.4 |
| 3 | 26.0 | 24.3 | 21.5 | 22.5 | 25.4 | 21.5 | 18.9 |
| 7 | 29.2 | 26.4 | 28.5 | 23.8 | 25.0 | 21.4 | 22.5 |
| MIG after x d | | | | | | | |
| 1 | 1.0 | 1.2 | 1.8 | 3.2 | 0.9 | <0.2 | <0.2 |
| 3 | <0.2 | <0.2 | <0.2 | <0.2 | <0.2 | <0.2 | <0.2 |
| 7 | <0.2 | <0.2 | <0.2 | <0.2 | <0.2 | <0.2 | <0.2 |

*The values given are averages of two independent productions of the film composites in each case.

It is shown that, using the adhesive formulations according to the invention, after storage for 1 day a lower migration value for PAAs is achieved than with the adhesive formulations not according to the invention.

1.-12. (canceled)

13. A method for the production of migrate-free adhesive bonds between substrates comprising applying an adhesive composition between two substrates, wherein the adhesive composition comprises an isocyanate-terminated polyurethane prepolymer having tertiary amino groups and structural elements of the formula —CH₂—CH₂—O—.

14. The method according to claim 13, wherein the substrates are films for food packaging.

15. The method according to claim 14, wherein the film composites obtained are migrate-free after no more than three days according to the requirements of section 64 LFGB.

16. The method according to claim 13, wherein the isocyanate-terminated polyurethane prepolymer has an NCO content of from 5 to 20 wt. % and a nominal average functionality of from 2 to 3.

17. The method according to claim 13, wherein the isocyanate-terminated polyurethane prepolymer is produced using a polyisocyanate having an NCO content of from 21 to 50 wt. % and a nominal average functionality of from 2 to 3.5.

18. The method according to claim 13, wherein the isocyanate-terminated polyurethane prepolymer is produced using a polyol or polyol mixture which comprises at least one tertiary amino group-containing polyether.

19. The method according to claim 13, wherein the isocyanate-terminated polyurethane prepolymer is produced using a polyol which comprises a tertiary amino group-containing polyether having a number average molecular weight Mₙ of from 320 to 20000 g/mol and a nominal functionality of from 2 to 4.5.

20. The method according to claim 13, wherein the isocyanate-terminated polyurethane prepolymer is produced using a polyol comprising a tertiary amino group-containing polyether with a hydroxyl value of 40 to 300 mg KOH/g.

21. The method according to claim 13, wherein the isocyanate-terminated polyurethane prepolymer is produced using a polyol wherein at least one of the polyols comprises structural elements of the formula —CH₂—CH₂—O—, and is produced using an ethylene oxide monomer.

22. An adhesive or plaster system comprising an isocyanate-terminated polyurethane prepolymer having tertiary amino groups and structural elements of the formula —CH₂—CH₂—O—.

23. The adhesive system according to claim 22, wherein the adhesive system further comprises a polyol or polyol mixture which also comprises structural elements of the formula —CH₂—CH₂—O—.

24. The adhesive or plaster system according to claim 22, wherein the adhesive or plaster system is for wound closure and/or care.

25. The adhesive or plaster system according to claim 22, wherein the isocyanate-terminated polyurethane prepolymer has an NCO content of from 5 to 20 wt. % and a nominal average functionality of from 2 to 3.

26. The adhesive or plaster system according to claim 22, wherein the isocyanate-terminated polyurethane prepolymer is produced using a polyisocyanate having an NCO content of from 21 to 50 wt. % and a nominal average functionality of from 2 to 3.5.

27. The adhesive or plaster system according to claim 23, wherein the polyol or polyol mixture which comprises at least one tertiary amino group-containing polyether.

28. The adhesive or plaster system according to claim 23, wherein the polyol or polyol mixture comprises a tertiary amino group-containing polyether having a number average molecular weight Mₙ of from 320 to 20000 g/mol and a nominal functionality of from 2 to 4.5.

29. The adhesive or plaster system according to claim 23, wherein the polyol or polyol mixture comprises a tertiary amino group-containing polyether with a hydroxyl value of 40 to 300 mg KOH/g.

30. The adhesive or plaster system according to claim 23, wherein at least one of the polyols is produced using an ethylene oxide monomer.

31. A wound closure system comprising an isocyanate-terminated polyurethane prepolymer having tertiary amino groups and structural elements of the formula —CH₂—CH₂—O—.
